(19) United States
(12) Patent Application Publication
(10) Pub. No.: US 2011/0245746 A1
(43) Pub. Date: Oct. 6, 2011

(54) MEDICAL CAST AND SKIN PROTECTOR

(76) Inventors: Choong Ha Lee, Gyeonggi-do (KR); Jong Kun Han, Seoul (KR); Byeong Su Cheon, Ulsan (KR)

(21) Appl. No.: 13/126,733

(22) PCT Filed: Oct. 27, 2009
(86) PCT No.: PCT/KR09/06241

§ 371 Date: Apr. 28, 2011

(30) Foreign Application Priority Data
Oct. 28, 2008 (KR) 10-2008-0105676

(51) Int. Cl. A61F 5/00

(52) U.S. Cl. 602/12

(57) ABSTRACT

The present invention relates to a medical cast and a skin protector. More particularly, the present invention relates to: a medical cast wherein a hardening solution having hydraulicity is impregnated onto a casting fabric and hardened, the casting fabric being woven throughout by a mesh weaving method and having cuffs formed at a part corresponding to a joint area of the human body and at both end parts of the casting fabric so as to prevent a gap between the skin and the cast, a cutting line of small thickness densely woven lengthwise on one side of the casting fabric, and a strength reinforced strip of large thickness densely woven lengthwise on both sides of the cutting line; and a medical skin protector to which a skin protection strip of a soft material is attached to protect the skin from a cutter blade during cutting of the medical cast.

### BACKGROUND OF THE INVENTION

1. Field of the Invention

The present invention relates to an orthopedic cast and a skin protector that are used mainly for treatment of a fracture patient, and more particularly, to an orthopedic cast that is formed to be easily cut by a cutter upon cutting the cast in order to remove it and a skin protector that is located within the cast and is formed to prevent the patient's skin from contacting with a cutter blade and thus prevents skin damage such as burns.

2. Description of the Related Art

Conventionally, an orthopedic cast is made by impregnating thin tape type glass fibers or polyester fibers into a hydraulic hardening material solution and is wound with several folds in a fractured portion of a patient and thus a winding pressure or thickness of the cast varies irregularly according to a surgical operation method of a surgical operator, whereby the patient receives a partially different pressure, not a uniform pressure and thus suffers trouble in blood circulation, a surgical operation is complicated, and a stockinet and wadding bandage are used for a skin protector attached in the inside of the cast, and thus have no ventilation property, whereby a problem of itching, stuffiness, uncleanness, and odor occurs.

In order to solve the above problems, in Korean Patent No. 10-0792252, entitled “medical cast and skin protector” previously applied by an applicant, the medical cast and skin protector are produced to have a trapezoid shape and a jar shape of a net structure by knitting using spandex and polyester yarns, are impregnated into a hydraulic hardening material solution and the medical cast and skin protector are used on a fractured portion of a patient, such that the medical cast and skin protector can be easily applied to an irregular body portion of the patient and provide good flexibility, ventilation property, and comfortable feeling.

However, in the medical cast and skin protector disclosed in Korean Patent No. 10-0792252, a large amount of load is applied to the cutter when using the cutter in order to remove the cast and thus much heat occurs in a cutter blade, whereby the patient's skin may suffer burns and particularly, the patient may suffer burns more seriously when a strength reinforced zone of the medical cast having a high strength is cut.

### SUMMARY OF THE INVENTION

### Technical Problem

The present invention has been made in view of the above problems, and provides an orthopedic cast including a cast base formed in a substantially cylindrical net form, and including tightening portions for preventing space from being formed between a patient's skin and the cast base formed in a portion corresponding to a joint of the patient and formed in both ends of the cast base; a cutting zone having a small thickness and densely formed along a length direction of the cast base; and strength reinforced zones each having a large thickness and densely formed at both sides of the cutting zone in the length direction of the cast base, wherein the cast base is treated by impregnating it into a hydraulic hardening material solution; and a skin protector having a skin protecting band of a flexible material attached to one surface of the skin protector in order to protect the patient's skin from being cut by a cutter blade upon cutting the cast.

### Technical Solution

In accordance with an aspect of the present invention, an orthopedic cast includes: a cast base formed in a substantially cylindrical net form, and including tightening portions for preventing space from being formed between a patient's skin and the cast base formed in a portion corresponding to a joint of the patient and formed in both ends of the cast base; a cutting zone having a small thickness and densely formed along a length direction of the cast base; and strength reinforced zones having a large thickness and densely formed at both sides of the cutting zone in the length direction of the cast base, wherein the cast base is impregnated into a hydraulic hardening material solution.

Preferably, the cutting zone is formed in a width of 1 to 30 mm.

Preferably, the strength reinforced zone is formed in a width of 0.5 to 2 cm.

In accordance with another aspect of the present invention, a skin protector is provided for use in an orthopedic surgery, wherein the skin protector is formed in an substantially cylindrical net form and includes tightening portions for preventing space from being formed between a patient's skin and the skin protector formed in a portion corresponding to a joint of the patient and formed in both ends of the skin protector; and a skin protecting band of a flexible material attached to one surface of the skin protector in order to protect the skin from being cut by a cutter blade upon cutting the cast.

Preferably, the skin protecting band is made of a foam material of a closed cell structure.

Preferably, the skin protecting band is made of a gel material.

Preferably, the skin protecting band has a thickness of 1 to 30 mm and a width of 1 to 8 cm.

### Advantageous Effects

In an orthopedic cast according to an exemplary embodiment of the present invention, as a cutting zone having a small thickness is formed along a length direction of the cast between strength reinforced zones each having a large thickness, the cast can be easily cut by a cutter when removing the cast.

Further, in a skin protector according to an exemplary embodiment of the present invention, as a skin protecting band of a foam or gel material is attached to the skin protector, a cutter blade is prevented from directly contacting with a patient's skin, and heat generated by the cutter is prevented from being transferred to the skin, whereby skin damage due to burns can be prevented.

### BRIEF DESCRIPTION OF THE DRAWINGS

FIG. 1 is a perspective view illustrating a state before application of a cast base and a skin protector for an orthopedic surgery according to an exemplary embodiment of the present invention; and

FIG. 2 is a picture illustrating a state of cutting an orthopedic cast with a cutter according to an exemplary embodiment of the present invention.

### DESCRIPTION OF REFERENCE NUMERALS INDICATING PRIMARY ELEMENTS IN THE DRAWINGS

1: cast base
2: skin protector
3: cutting zone
4: strength reinforced zone
5: skin protecting band
6: tightening portion
7: wrist (ankle) tightening portion
8: cutter
9: cutter blade

### DETAILED DESCRIPTION OF EXEMPLARY EMBODIMENTS

Hereinafter, exemplary embodiments of a cast base and a skin protector for an orthopedic surgery according to an exemplary embodiment of the present invention are described in detail through the accompanying drawings. The views in the drawings are schematic views only, and are not intended to be to scale or correctly proportioned.

The same reference numbers are used throughout the drawings to refer to the same or like parts. Detailed descriptions of well-known functions and structures incorporated herein may be omitted to avoid obscuring the subject matter of the present invention.

While the present invention may be embodied in many different forms, specific embodiments of the present invention are shown in drawings and are described herein in detail, with the understanding that the present disclosure is to be considered as an exemplification of the principles of the invention and is not intended to limit the invention to the specific embodiments illustrated.

### Exemplary Embodiments

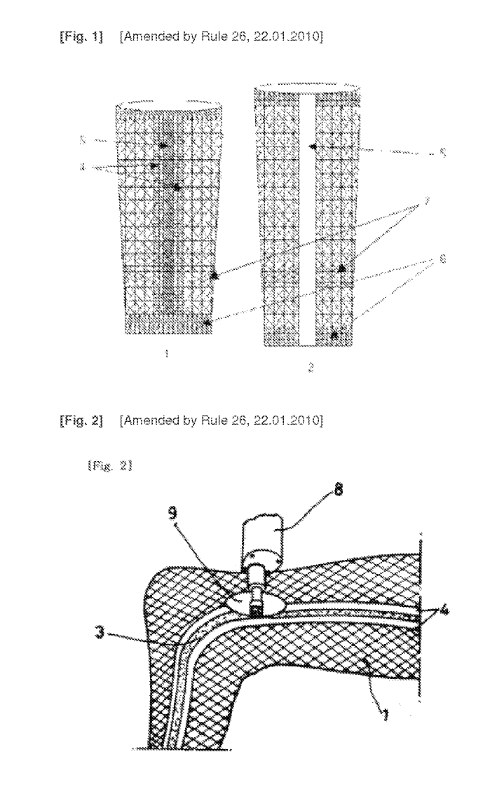

FIG. 1 is a perspective view illustrating a state before application of a cast base and a skin protector for an orthopedic surgery according to an exemplary embodiment of the present invention, and FIG. 2 is a picture illustrating a state of cutting an applied orthopedic cast with a cutter according to an exemplary embodiment of the present invention.

Referring to FIG. 1, provided is a cast base 1 of a cylindrical net form in which one end thereof has a small diameter and the other end thereof has a larger diameter, in order for the cast base 1 to closely contact with a patient's skin by improving flexibility at a joint of the patient such as a wrist or an ankle, densely formed tightening portions 6 are formed in both ends of the cast base 1, and when the cast base 1 is covered in a fractured portion of the patient, a densely formed wrist (ankle) tightening portion 7 is formed in a portion corresponding to the patient's wrist or ankle joint.

Further, provided is a skin protector 2 of a cylindrical net form in which one end thereof has a small diameter and the other end thereof has a larger diameter, and tightening portions 6 are formed in both ends of the skin protector 2, in order for the skin protector 2 to closely contact with the patient's skin by improving flexibility at a joint of the patient such as a wrist or an ankle, and when the skin protector 2 is covered in the fractured portion of the patient, a densely formed wrist (ankle) tightening portion 7 is formed in a portion corresponding to the patient's wrist or ankle joint.

In this case, it is preferable to manufacture the cast base 1 and the skin protector 2 by knitting using polyester and spandex yarns having excellent flexibility, and the cast base 1 and the skin protector 2 are manufactured in a cylindrical shape in which one ends of the cast base 1 and the skin protector 2 have a small diameter and the other ends of the cast base 1 and the skin protector 2 have a larger diameter and are formed in an substantially cylindrical net form to sustain a uniform ventilation property and uniform strength.

Further, in order to easily perform absorption and discharge of moisture, it is preferable to perform a water repellent treatment on a surface of the skin protector 2 or to knit the skin protector 2 using high water repellent yarns.

The cast base 1 to which a hydraulic hardening material solution is treated and the skin protector 2 are wound separately, packed, and distributed, and when using the cast base 1 and the skin protector 2, after placing the wound skin protector 2 in a fractured portion of a patient and spreading the skin protector 2, the wound cast base 1 treated by a hydraulic hardening material solution is soaked in water and then the cast base 1 is spread on the skin protector 2, and as the cast base 1 is hardened, the cast base 1 is attached to the fractured portion of the patient and thus a surgical operation is complete.

FIG. 1 illustrates the cast base 1 formed in a cylindrical net form, and in order to increase an impregnation rate of a hydraulic hardening material solution and to increase flexibility, it is preferable to knit using covering yarns in which spandex yarns are covered with polyester yarns having bulkiness, and as the cast base 1 has a laterally uniform strand quantity, the cast base 1 sustains uniform strength.

In this case, “bulkiness” is a property that externally looks like thick and soft due to fine hairs such as woolen yarns.

Further, the strand quantity of the cast base 1 may be adjusted according to a requested degree of strength, and the cast base 1 has excellent flexibility and an excellent restoring force by a network structure and a structure of spandex covering yarns.

When removing the cast base 1, the hardened cast is cut using a cutter blade 9 of a cutter 8 having a circular saw blade, and in order to guide a moving direction of the cutter blade 9, a cutting zone 3 having a width of 1 to 30 mm is densely formed in a small thickness along a length direction of the cast base 1 and the cutting zone 3 is preferably manufactured with a different color in order to be easily found.

In this case, if a width of the cutting zone 3 is smaller than 1 mm, it is difficult to perform a cutting operation, and if a width of the cutting zone 3 is larger than 30 mm, strength and durability of the cast base 1 is deteriorated.

In order to increase a strength of the cast base 1, a pair of strength reinforced zones 4 densely formed in a width of about 0.5 to 2 cm in symmetry about the cutting zone 3 are each formed in a length direction of the cast base 1 and thus the cast base 1 can sustain a high strength. In order words, the cutting zone 3 having a thickness smaller than that of the cast base 1 is formed, and the strength reinforced zones 4 having a thickness larger than that of the cast base 1 are formed at both sides of the cutting zone 3.

Therefore, the strength reinforced zones 4 are formed to have a large thickness and are formed at both sides of the cutting zone 3 of a small thickness, and thus upon cutting, when the cutter blade 9 escapes from the cutting zone 3, the cutter blade 9 is caught by the strength reinforced zone 4 and thus the strength reinforced zone 4 guides a moving direction of the cutter 8, whereby a cutting operation of the cast is easily performed.

As shown in FIG. 1, only the wrist (ankle) tightening portion 7 may be formed or an elbow (knee) tightening portion (not shown) may be formed in an upper part of the wrist (ankle) tightening portion 7.

Further, the cast base 1 may be formed in a rectangular form and may be formed in a truncated conical form or a jar form to cover easily in a human body portion, as shown in FIG. 1.

In this case, the cast base 1 of a jar form is formed to have different widths along a length direction of the cast base 1 and can be easily applied corresponding to various body sizes.

The skin protector 2 according to an exemplary embodiment of the present invention has an excellent ventilation property, excellent flexibility, and an excellent restoring force by knitting using high water repellent yarns such as polyolefin or Gore-Tex yarns and spandex covering yarns.

Further, propagation of bacteria is prevented by an antibacterial treatment, thus itching problem is solved, a surgical operation can be simply performed, an excellent comfortable feeling can be obtained, a clean state can be sustained, and a quick healing effect can be obtained.

In order to protect the skin from the hard cast, by knitting to have a dual structure, an internal fabric of a soft material contacts with the skin and an outer fabric of another material contacts with the cast base 1 and thus a more comfortable feeling can be provided and the cast base 1 may be formed by adjusting the strand quantity according to a required flexible degree.

Here, in order to prevent that the cutter blade 9 of the cutter 8 directly contacts with the skin when removing the cast and that heat generated by a friction in the cutter blade 9 is transferred to the skin, a skin protecting band 5 is made of a flexible material of a width of 1 to 8 cm and a thickness of 1 to 30 mm and is attached to a portion in which the skin protector 2 is to be cut along a length direction of the skin protector 2 using a method such as fusing and bonding, thereby stability of the skin protector can be provided, and upon performing a surgical operation, it is preferable to apply the cast base 1 to correspond the skin protecting band 5 of the skin protector 2 to the cutting zone 3 of the cast base 1.

In this case, if a width of the skin protecting band 5 is smaller than 1 cm, the skin cannot be appropriately protected upon cutting, and if a width of the skin protecting band 5 is larger than 8 cm, a patient feels stuffy.

Further, if a thickness of the skin protecting band 5 is smaller than 1 mm, it is difficult to safely protect the skin from the cutter blade 9, and if a thickness of the skin protecting band 5 is larger than 30 mm, it is difficult to closely contact the cast with a fractured portion.

Further, the skin protecting band 5 can be formed in a foam form or a gel form, and the skin protecting band 5 of a foam form has a structure of a porous foam made of a polymer material such as polyurethane and polyolefin, and a cell within the porous foam is a closed cell, not an open cell and preferably has a form of a polymer foam that prevents absorption of moisture or other contamination. When foam of an open cell is used, if the cast is soaked in water by mistake, water penetrated into the foam cannot come out from the foam, and thus the patient feels itching and the foam may be contaminated.

Here, an open cell indicates a cell of a state in which cells formed within the foam are connected by carbon dioxide generated during a foaming process, and a closed cell indicates a cell of a state in which cells within the foam are independently formed.

When the skin protecting band 5 is formed in a gel form, the skin protecting band 5 is made of a polymer material such as polyurethane or polyolefin to have flexibility such as a skin of a human body and thus the patient's skin can be safely protected from the cutter blade 9.

That is, because the cutter 8 used for removing the cast has a circular saw blade as the cutter blade 9 and has a characteristic that easily cuts a hard material but does not easily cut a soft and durable material, and the skin protecting band 5 of a foam or gel form is not easily cut by the cutter 8, the skin protecting band 5 prevents a direct contact between the cutter blade 9 and the patient's skin and prevents heat of the cutter blade 9 from being transferred to the skin.

In this case, as in the cast base 1, in the skin protector 2, an elbow (knee) tightening portion (not shown) may be formed in an upper part of the wrist (ankle) tightening portion 7.

Further, the skin protector 2 may be formed in a rectangular form, and it is preferable to knit the skin protector 2 in a truncated conical form or a jar form in order to easily cover a body portion.

In this case, a jar form of skin protector 2 formed in different widths in a length direction thereof can be applied to easily correspond to various body sizes.

FIG. 2 illustrates a state of removing a cast after application of a cast and a skin protector and illustrates a case where a fractured portion is an ankle portion.

It is preferable that the skin protector 2 and the cast base 1 in which a hydraulic hardening material solution is impregnated are wound separately, packed, and distributed, and application of the skin protector 2 is performed by putting and spreading the wound skin protector 2 in the fractured portion, and the cast base 1 in which a hydraulic hardening material solution is impregnated is soaked in water, and is spread on the skin protector 2 after putting and spreading the skin protector 2.

When removing the cast, the hardened cast base 1 is cut along the cutting zone 3 using the cutter 8, and the cutter blade 9 can easily perform a cutting operation while preventing escape from the cutting zone 3 because the cutting zone 3 of the cast base 1 is in a hardened state by a hydraulic hardening material solution, and the cutting zone 3 has a thickness smaller than the strength reinforced zones 4 in both sides thereof.

Further, because the skin protecting band 5 is formed at the outside of the skin protector 2 corresponding to the cutting zone 3 of the cast base 1, a doctor can remove the cast by performing a cutting operation without difficulty, and a patient can feel no fear for the cutter blade 9 rotating in a high speed and can sustain a psychologically stable state while performing a cutting operation.

### INDUSTRIAL APPLICABILITY

In a cast base for an orthopedic surgery according to an exemplary embodiment of the present invention, as a cutting zone having a small thickness is formed between strength reinforced zones having a large thickness in a length direction of the cast base, the cast base can be easily removed by a cutter when removing the cast base.

Further, in a skin protector according to an exemplary embodiment of the present invention, as a skin protecting band of a foam or gel material is attached to the skin protector, a cutter blade is prevented from directly contacting with a patient's skin, and heat generated in the cutter is prevented from being transferred to the skin, whereby skin damage due to burns can be prevented.

1. An orthopedic cast comprising a cast base formed in a substantially cylindrical net form, and comprising:
tightening portions for preventing space from being formed between a patient's skin and the cast base formed in a portion corresponding to a joint of the patient and formed in both ends of the cast base;
a cutting zone having a small thickness and densely formed along a length direction of the cast base; and
strength reinforced zones having a large thickness and densely formed at both sides of the cutting zone in the length direction of the cast base, wherein the cast base is impregnated into a hydraulic hardening material solution.

2. The orthopedic cast of claim 1, wherein the cutting zone is formed in a width of 1 to 30 mm.

3. The orthopedic cast of claim 1, wherein the strength reinforced zone is formed in a width of 0.5 to 2 cm.

4. A skin protector for use in an orthopedic surgery formed in a substantially cylindrical net form and includes:
tightening portions for preventing space from being formed between a patient's skin and the skin protector formed in a portion corresponding to a joint of the patient and formed in both ends of the skin protector; and
a skin protecting band of a flexible material attached to one surface of the skin protector in order to protect the skin from being cut by a cutter blade upon cutting the cast.

5. The skin protector of claim 4, wherein the skin protecting band is made of a foam material.

6. The skin protector of claim 5, wherein the foam material has a closed cell structure.

7. The skin protector of claim 4, wherein the skin protecting band is made of a gel material.

8. The skin protector of claim 4, wherein the skin protecting band has a thickness of 1 to 30 mm and a width of 1 to 8 cm.

9. The orthopedic cast of claim 2, wherein the strength reinforced zone is formed in a width of 0.5 to 2 cm.
